(19) United States
(12) Patent Application Publication
(10) Pub. No.: US 2022/0240790 A1
(43) Pub. Date: Aug. 4, 2022

(54) SYSTEMS AND METHODS FOR NON-CONTACT HEART RATE MONITORING

(71) Applicant: Covidien LP, Mansfield, MA (US)

(72) Inventors: Philip C. SMIT, Hamilton (GB); Paul S. ADDISON, Scotland (GB)

(21) Appl. No.: 17/588,723

(22) Filed: Jan. 31, 2022

Related U.S. Application Data
(60) Provisional application No. 63/145,403, filed on Feb. 3, 2021.

(51) Int. Cl. A61B 5/0205; A61B 5/024; A61B 5/08

(52) U.S. Cl. CPC A61B 5/0205; A61B 5/0816; A61B 5/02433

(57) ABSTRACT

Methods and systems for non-contact monitoring of a patient to determine cardiac information about the patient, for example, heart rate or pulse. The methods and systems can additionally monitor respiratory parameters if desired. The methods and systems of this disclosure utilize light intensity of reflected features (e.g., IR, UV, RBG, or other light) to determine the cardiac parameter and can also utilize the light intensity of reflected features to determine the respiratory parameter. The same or a different respirator parameter can be determined by depth (distance) information.

### CROSS-REFERENCE

This application claims priority to U.S. provisional application No. 63/145,403 filed Feb. 3, 2021 and entitled SYSTEMS AND METHODS FOR NON-CONTACT HEART RATE MONITORING, which is incorporated herein by reference for all purposes.

### BACKGROUND

Many conventional medical monitors require attachment of a sensor to a patient in order to detect physiologic signals from the patient and transmit detected signals through a cable to the monitor. These monitors process the received signals and determine vital signs such as the patient's pulse rate, respiration rate, and arterial oxygen saturation. For example, a pulse oximeter is a finger sensor that may include two light emitters and a photodetector. The sensor emits light into the patient's finger and transmits the detected light signal to a monitor. The monitor includes a processor that processes the signal, determines vital signs (e.g., pulse rate, respiration rate, arterial oxygen saturation), and displays the vital signs on a display.

Other monitoring systems include other types of monitors and sensors, such as electroencephalogram (EEG) sensors, blood pressure cuffs, temperature probes, air flow measurement devices (e.g., spirometer), and others. Some wireless, wearable sensors have been developed, such as wireless EEG patches and wireless pulse oximetry sensors.

Video-based monitoring is a new field of patient monitoring that uses a remote video camera to detect physical or physiological attributes of the patient. This type of monitoring may also be called “non-contact” monitoring in reference to the remote video sensor, which does not contact the patient.

### SUMMARY

The present disclosure is directed to methods and systems for non-contact monitoring of a patient to determine cardiac information about the patient, particularly, heart rate or pulse. The methods and systems can additionally monitor respiratory parameters if desired.

The methods and systems of this disclosure utilize light intensity of reflected features (e.g., IR, UV, RBG, or other light) to determine cardiac parameter such as heart rate or pulse. The methods and systems of this disclosure additionally optionally utilize light intensity of reflected features (e.g., IR, UV, RBG, or other light) to determine a respiratory parameter such as respiratory rate. The methods and systems of this disclosure additionally optionally utilize depth (distance) information between the camera(s) and the patient to determine a respiratory parameter such as respiratory rate.

The systems and methods receive a light intensity signal from a feature projected onto the patient, such as an IR feature, and from that calculate the heart rate or pulse. The systems and methods can also utilize the light intensity signal to calculate respiratory parameter(s) such as respiration rate, tidal volume, minute volume, and other parameters such as motion or activity. Still further, in some embodiments, the methods and systems utilize a video signal of the patient and from that extract a distance or depth signal to calculate respiratory parameter(s) from the depth signal. The respiratory parameter(s) from the two signals can be combined or compared to provide a qualified output respiratory parameter.

One particular embodiment described herein is method of monitoring a patient by a non-contact patient monitoring system in a region of interest (ROI), over time. The method includes determining a cardiac parameter of the patient using reflected light intensity information in the ROI, over time; this is by projecting a light feature onto a surface of the patient in the ROI over time, measuring a first reflected light intensity from the light feature at a first time, measuring a second reflected light intensity from the light feature at a second time subsequent to the first time, comparing the first reflected light intensity and the second reflected light intensity to determine a change in profile of the surface over time, and obtaining a pattern in the change in surface profile over time and correlating the pattern to the cardiac parameter.

In some embodiments, the method also includes determining a respiratory parameter of the patient using reflected light intensity information in a second ROI, over time, from the patient, which is determined by projecting a light feature onto the surface of the patient in the second ROI over time, measuring a third reflected light intensity from the light feature at a third time, measuring a fourth reflected light intensity from the light feature at a fourth time subsequent to the third time, comparing the third reflected light intensity and the fourth reflected light intensity to determine a change in profile of the surface over time, and obtaining a pattern of the of change in surface profile over time and correlating the pattern to the respiratory parameter.

Additionally or alternately, in some embodiments, the method includes determining the respiratory parameter of the patient using depth information, and comparing the respiratory parameter determined using depth information to the respiratory parameter determined using light intensity, if applicable.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Other embodiments are also described and recited herein.

### BRIEF DESCRIPTION OF THE DRAWING

FIG. 1 is a schematic diagram of an example non-contact patient monitoring system according to various embodiments described herein.

FIG. 2 is a photograph of a patient with a reference grid superimposed thereon.

FIG. 3A and FIG. 3B are schematic diagrams showing two light intensity measurement examples using the example non-contact patient monitoring system of FIG. 1.

FIG. 4A is a graphical representation of data obtained by a non-contact patient monitoring system according to various embodiments described herein; and FIG. 4B is a graphical representation of the data of FIG. 4A combined to provide a signal.

FIG. 5 is representation of heart rate wavelet-based analysis over time.

FIG. 6A is a graphical representation of respiratory data obtained from light intensity according to various embodiments described herein; and FIG. 6B is a graphical representation of respiratory data obtained from depth data according to various embodiments described herein.

FIG. 7 is a stepwise method of an example method of using a non-contact patient monitoring system according to various embodiments described herein to obtain a respiratory parameter from two measurement sources.

FIG. 8 is a stepwise method of an example method of using a non-contact patient monitoring system according to various embodiments described herein.

FIG. 9 is a block diagram of a computing device, a server, and an image capture device according to various embodiments described herein.

### DETAILED DESCRIPTION

As described above, the present disclosure is directed to medical monitoring, and in particular, non-contact, video-based monitoring of a cardiac parameter (e.g., heart rate, or pulse) and optionally one or more respiratory parameters, including respiration rate, tidal volume, minute volume, and other parameters such as motion or activity.

Systems and methods are described here that receive a light intensity signal from a patient comprised of individual light intensity data points reflected from projected features in a relevant area (such as a patient's forehead or chest) and calculate a cardiac parameter from the combined individual data points. The systems and methods may also calculate a respiratory parameter from the light intensity signal from projected features in a relevant area (such as a patient's chest) and calculate the respiratory parameter from the data points. The systems and methods may also calculate the respiratory parameter from a video signal view of the patient, by identifying a physiologically relevant area within the video image (such as a patient's chest), and extracting a distance or depth signal from the relevant area. This measurement can be compared to the respiratory parameter calculated from the light intensity signal.

The light intensity signal and depth signal are detected by a camera system that does not contact the patient. With appropriate selection and filtering of the light intensity signal detected, the heart rate can be calculated. Additionally, the light intensity signal can be appropriately selected and filtered to calculate a respiratory parameter. Further, the same camera system or a different camera system can be used to detect a depth or distance between the camera system and the patient, which can be used to calculate the respiratory parameter.

In such a manner, useful vital sign measurements (e.g., heart rate and a respiratory parameter) can be determined without placing a detector in physical contact with the patient. This approach has the potential to improve patient mobility and comfort, along with many other potential advantages discussed below.

Remote sensing of a patient with video-based monitoring systems presents several challenges. One challenge is due to motion or movement of the patient. The problem can be illustrated with the example of pulse oximetry. Conventional pulse oximetry sensors include two light emitters and a photodetector. The sensor is placed in contact with the patient, such as by clipping or adhering the sensor around a finger, toe, or ear of a patient. The sensor's emitters emit light of two particular wavelengths into the patient's tissue, and the photodetector detects the light after it is reflected or transmitted through the tissue. The detected light signal, called a photoplethysmogram (PPG), modulates with the patient's heartbeat, as each arterial pulse passes through the monitored tissue and affects the amount of light absorbed. The detected PPG signal is based on a color change of the light, which is directly related to the amount of light absorbed. Movement of the patient can interfere with this contact-based oximetry, introducing noise into the PPG signal due to compression of the monitored tissue, disrupted coupling of the sensor to the finger, pooling or movement of blood, exposure to ambient light, and other factors. Modern pulse oximeters use filtering algorithms to remove noise introduced by motion and to continue to monitor the pulsatile arterial signal.

However, movement in non-contact pulse oximetry creates different complications, due to the extent of movement possible between the patient and the camera. Because the camera is remote from the patient, the patient may move towards or away from the camera, creating a moving frame of reference, or may rotate with respect to the camera, effectively morphing the region that is being monitored. Thus, the monitored tissue can change morphology within the image frame over time. This freedom of motion of the monitored tissue with respect to the detector introduces new types of motion noise into the video-based signals.

Another challenge is ambient light. In this context, “ambient light” means surrounding light not emitted by components of the camera or the monitoring system. In contact-based pulse oximetry, the desired light signal is the reflected and/or transmitted light from the light emitters on the sensor, and ambient light is entirely noise. The ambient light can be filtered, removed, or avoided in order to focus on the desired signal. In contact-based pulse oximetry, contact-based sensors can be mechanically shielded from ambient light, and direct contact between the sensor and the patient also blocks much of the ambient light from reaching the detector. By contrast, in non-contact pulse oximetry, the desired physiologic signal is generated or carried by the ambient light source; thus, the ambient light cannot be entirely filtered, removed, or avoided as noise. Changes in lighting within the room, including overhead lighting, sunlight, television screens, variations in reflected light, and passing shadows from moving objects all contribute to the light signal that reaches the camera. Even subtle motions outside the field of view of the camera can reflect light onto the patient being monitored.

Non-contact monitoring can deliver significant benefits over contact monitoring if the above-discussed challenges can be addressed. Some non-contact monitoring can reduce cost and waste by reducing use of disposable contact sensors, replacing them with reusable camera systems. Non-contact monitoring may also reduce the spread of infection, by reducing physical contact between caregivers and patients. Video cameras can improve patient mobility and comfort, by freeing patients from wired tethers or bulky wearable sensors. In some cases, these systems can also save time for caregivers, who no longer need to reposition, clean, inspect, or replace contact sensors.

The present disclosure describes methods and systems for non-contact monitoring of a patient to determine a cardiac parameter such as heart rate or pulse rate and optionally also determine respiratory parameter(s) such as respiration rate, tidal volume, minute volume, and other parameters such as motion and activity. The systems and methods receive a light intensity signal from a feature projected onto the patient and calculate the cardiac parameter from the reflected light intensity signal as it changes over time (e.g., a pattern); the reflected light intensity signal is independent of any color change that may occur. The light intensity signal can also be used to calculate respiratory parameter(s). In some embodiments, the systems and methods also receive a video signal from the patient and from that extract a distance or depth signal to again calculate the respiratory parameter(s). The parameter(s) from the two signals can be combined or compared to provide a qualified output parameter.

A projector is used to project features (e.g., in the form of dots, pixels, etc.) onto the desired surface area to be monitored; these projected features are monitored over time by at least one camera for changes in the light intensity reflected by the surface. Each projected feature may be monitored, or less than all the features in the ROI may be monitored. When two cameras set at a fixed distance apart are used, they offer stereo vision due to the slightly different perspectives of the scene from which distance information is extracted. When distinct features are present in the scene, the stereo image algorithm can find the locations of the same features in the two image streams.

In the following description, reference is made to the accompanying drawing that forms a part hereof and in which is shown by way of illustration at least one specific embodiment. The following description provides additional specific embodiments. It is to be understood that other embodiments are contemplated and may be made without departing from the scope or spirit of the present disclosure. The following detailed description, therefore, is not to be taken in a limiting sense. While the present disclosure is not so limited, an appreciation of various aspects of the disclosure will be gained through a discussion of the examples, including the figures, provided below. In some instances, a reference numeral may have an associated sub-label consisting of a lower-case letter to denote one of multiple similar components. When reference is made to a reference numeral without specification of a sub-label, the reference is intended to refer to all such multiple similar components.

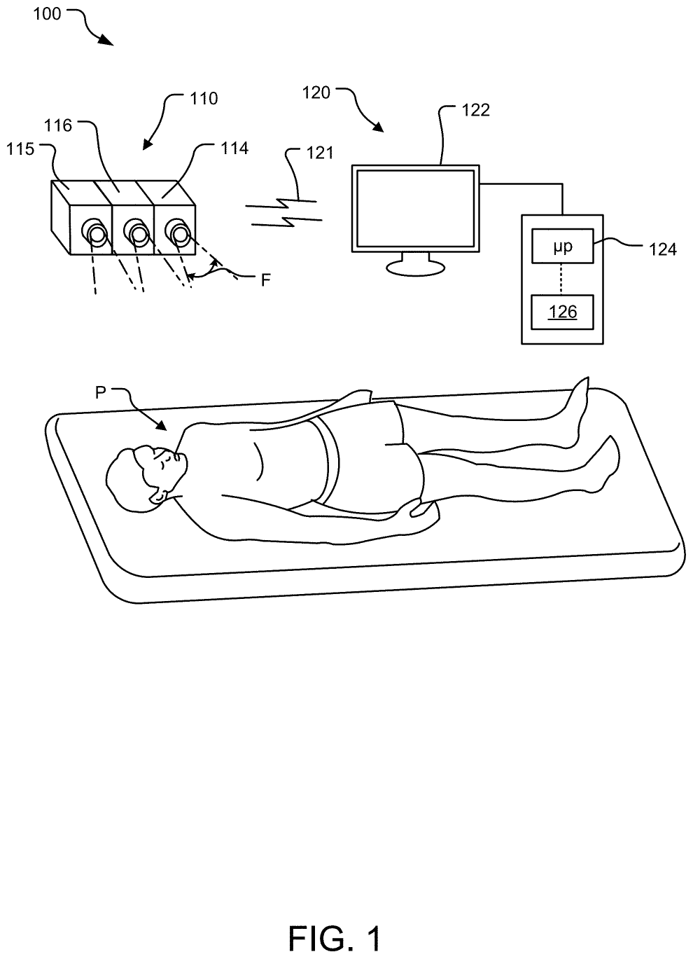

FIG. 1 shows a non-contact patient monitoring system 100 and a patient P according to an embodiment of the invention. The system 100 includes a non-contact detector system 110 placed remote from the patient P. In this embodiment, the detector system 110 includes a camera system having a first camera 114 and a second camera 115; in other embodiments, only one camera may be present in the detector system 110. An example of a suitable camera 114 and/or camera 115 is a Kinect camera from Microsoft Corp. (Redmond, Wash.) or a RealSense™ D415, D435 or D455 camera from Intel Corp. (Santa Clara, Calif.).

The detector system 110 is remote from the patient P, in that it is spaced apart from and does not physically contact the patient P. The detector system 110 includes a detector, typically as part of the cameras 114, 115, exposed to a field of view F that encompasses at least a portion of the patient P. The two cameras 114, 115 typically have the same field of view F, in order to provide stereo vision, although in other embodiments the two cameras 114, 115 may have different fields of view, e.g., one camera focused on the patient's torso and the other camera focused on the patient's head.

The monitoring system 100 includes a projector 116 that generates a sequence of individual features (e.g., dots, crosses or Xs, lines, individual pixels, etc.) onto the ROI. The features may be visible light, UV light, infrared (IR) light, etc. The IR may be, e.g., near infrared (NIR), short wave infrared (SWIR), midwave infrared (MWIR), or long wave infrared (LWIR). In an embodiment, each image or feature projected by the projector 116 includes a two-dimensional array or grid of pixels, and each pixel may include three color components—for example, red, green, and blue. A measure of one or more color components of one or more pixels over time is referred to as a “pixel signal,” which is a type of light intensity signal. These color components are different than and distinct from any color change observed due to absorbance of the light signal, as in PPG. In another embodiment, when the projector 116 projects an IR feature, which is not visible to a human eye, the detector system 110 includes an infrared (IR) sensing feature. In another embodiment, the projector 116 projects a UV feature. In yet other embodiments, other modalities including millimeter-wave, hyper-spectral, etc., may be used.

The projector 116 may alternately or additionally project a featureless intensity pattern (e.g., a homogeneous, a gradient or any other pattern that does not necessarily have distinct features). In some embodiments, the projector 116, or more than one projector, can project a combination of a feature-rich pattern and featureless patterns on to the ROI.

The projector may be part of the detector system 110 or the overall monitoring system 100, in that it is separate from the detector system 110. In some embodiments, there may be more than one projector. For one projector 116 or multiple projectors, the emission power may be dynamically controlled to modulate the light emissions, in a manner as commonly done for pulse-oximeters with LED light.

The projector 116 generates a sequence of features over time on the ROI from which is monitored and measured the reflectance of the features, as a reflected light intensity. A measure of the amount or brightness of light of all or a portion of the reflected features over time is referred to as a light intensity signal. The cameras 114, 115 detect the features from which this light intensity signal is determined.

The cameras 114, 115 have at a frame rate, which is the number of image frames taken per second (or other time period). Example frame rates include 20, 30, 40, 50, or 60 frames per second, greater than 60 frames per second, or other values between those. Frame rates of 20-30 frames per second produce useful signals, though frame rates above 100 or 120 frames per second are helpful in avoiding aliasing with light flicker (for artificial lights having frequencies around 50 or 60 Hz).

The cameras 114, 115 are aimed to have the features projected by the projector 116 to be in the ROI of the cameras 114, 115. The light from the projector 116 hitting the ROI surface is scattered/diffused in all directions; the diffusion pattern depends on the reflective and scattering properties of the surface. From this change in diffusion pattern, the light intensity and thus the distance between the cameras 114, 115 and the projected features can be detected by the cameras 114, 115.

The cameras 114, 115 measure the light intensity reflected from individual projected features from the ROI (e.g., exposed skin on the patient's face or forehead), as described further below, and the system 100 determines cardiac information from the changing, reflected, light intensity. Each projected feature may be monitored, or less than all the features in the ROI may be monitored. From the changing light intensity (e.g., reflected from the patient's face or torso), respiratory information can also be determined. The cardiac information and the respiratory information may be from the same or different ROIs; in other words, there may be different ROIs for the cardiac information and the respiratory information.

The detector system 110 can also include a depth sensing feature, such as a depth sensing camera, that can detect a distance between the detector system 110 and objects in the field of view F; one or both of the cameras 114, 115 may be a depth sensing camera in addition to detecting light reflection.

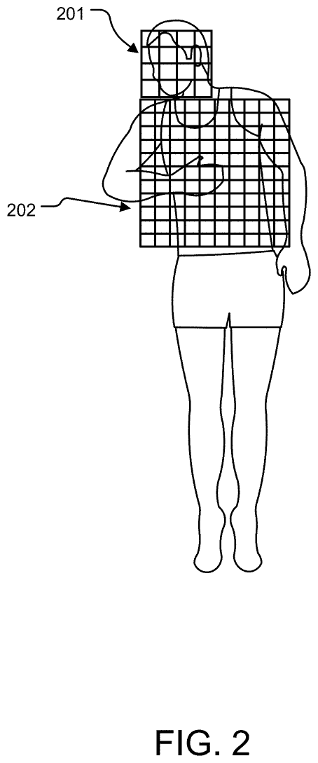

The depth information can be used to monitor an ROI on the patient and extract respiratory information from movements of the patient associated with, e.g., breathing. Accordingly, those movements, or changes of depth points within the ROI, can be used to determine, e.g., respiration rate, tidal volume, minute volume, effort to breathe, etc. Again, this ROI may be the same or different than the ROIs for the cardiac information and the light intensity-based respiration information. As an example, FIG. 2 shows a patient P (lying down) with a grid pattern superimposed onto the image of the patient P; the grid pattern is used to identify a series of ROIs. In this example, a first ROI 201 is on the patient's face for determining cardiac information and a second ROI 202 is on the chest of the patient P for determining respiratory information.

The distance from the ROI to the cameras 114, 115 can also be measured by the detector system 110. Generally, the detector system 110 detects a distance between the cameras 114, 115 and the surface within the ROI. The change in depth or distance within the ROI can represent movements of the patient, e.g., associated with breathing. For example, movement of the patient's chest toward the cameras 114, 115 as the patient's chest expands forward represents inhalation. Similarly, movement backward, away from the cameras 114, 115, occurs when the patient's chest contracts with exhalation. This movement forward and backward can be tracked to determine a respiration rate. Furthermore, this movement forward and backward can be integrated to determine a tidal volume.

In some embodiments, the monitoring system 100 determines a skeleton outline of the patient P to identify a point or points from which to extrapolate any of the ROIs. For example, a skeleton may be used to find a center point of a chest, shoulder points, waist points, and/or any other points on a body. These points can be used to determine the ROI. For example, the ROI may be defined by filling in area around a center point of the chest. Certain determined points may define an outer edge of an ROI, such as shoulder points. In other embodiments, instead of using a skeleton, other points are used to establish an ROI. For example, a face may be recognized, and a chest area inferred in proportion and spatial relation to the face. In other embodiments, the monitoring system 100 may establish the ROI around a point based on which parts are within a certain depth range of the point. In other words, once a point is determined that an ROI should be developed from, the system can utilize the depth information from the depth sensing cameras 114, 115 to fill out the ROI. For example, if a point on the chest is selected, depth information is utilized to determine the ROI area around the determined point that is a similar distance from the cameras 114, 115 as the determined point; this area is likely to be a chest.

In another example, the patient P may wear a specially configured piece of clothing that identifies points on the body such as forehead, shoulders or the center of the chest. The monitoring system 100 may identify those points by identifying the indicating feature of the clothing. Such identifying features could be a visually encoded message (e.g., bar code, QR code, etc.), or a brightly colored shape that contrasts with the rest of the patient's clothing, etc. In some embodiments, a piece of clothing worn by the patient may have a grid or other identifiable pattern on it to aid in recognition of the patient and/or their movement. In some embodiments, the identifying feature may be stuck on the clothing using a fastening mechanism such as adhesive, a pin, etc. For example, a small sticker or other indicator may be placed on a patient's shoulders and/or center of the chest that can be easily identified from an image captured by a camera. In some embodiments, the indicator may be a sensor that can transmit a light or other information to the cameras 114, 115 that enables its location to be identified in an image so as to help define an ROI. Therefore, different methods can be used to identify the patient and define an ROI.

The ROI size may differ according to the parameter(s) being monitored, and/or the distance of the patient from the detector system 110. For example, the ROI when monitoring the light intensity may have a smaller area than an ROI for measuring depth changes, e.g., because a larger data set may be desired for depth changes.

When measuring changes in depth, the ROI dimensions may vary linearly with the distance of the patient from the detector system 110. This ensures that the ROI scales accordingly with the patient and covers the same part of the patient regardless of the patient's distance from the cameras. This is accomplished by applying a scaling factor that is dependent on the distance of the patient (and the ROI) from the cameras 114, 115. In order to properly measure the depth changes, the actual size (area) of the ROI is determined and movements of that ROI are measured. The measured movements of the ROI and the actual size of the ROI are then used to calculate the respiratory parameter. Because a patient's distance from a camera can change, e.g., due to rolling or position readjustment, the ROI associated with that patient can appear to change in size in an image from a camera. However, using the depth sensing information captured by a depth sensing camera or other type of depth sensor, the system can determine how far away from the camera the patient (and their ROI) actually is. With this information, the actual size of the ROI can be determined, allowing for accurate measurements of depth change regardless of the distance of the camera to the patient.

In some embodiments, the monitoring system 100 may receive a user input to identify a starting point for defining an ROI. For example, an image may be reproduced on an interface, allowing a user of the interface to select a patient for monitoring (which may be helpful where multiple humans are in view of a camera) and/or allowing the user to select a point on the patient from which the ROI can be determined (such as a point on the chest). Other methods for identifying a patient, such as points on the patient or a superimposed grid on the patient, may also be used.

The detected light intensity measurements for the cardiac information, detected light intensity measurements for the respiratory information, and depth information for the second respiratory information from the ROI(s) are sent from the detector system 110 to a computing device 120 through a wired or wireless connection 121. The computing device 120 includes a display 122, a processor 124, and hardware memory 126 for storing software and computer instructions. The display 122 may be remote, such as a video screen positioned separately from the processor and memory. Other embodiments of the computing device 120 may have different, fewer, or additional components than shown in FIG. 1. In some embodiments, the computing device 120 may be a server. In other embodiments, the computing device of FIG. 1 may be additionally connected to a server. The captured data can be processed or analyzed at the computing device and/or at the server to determine the parameters of the patient P as disclosed herein.

Returning to FIG. 2, as indicated above, an ROI 201 is shown in the patient's face and an ROI 202 is shown on the patient's torso; both ROIs 201, 202 having a superimposed grid composed of multiple boxes. A series or array of reflection features (specifically, dots in FIG. 2) is seen in the ROI 201 and the ROI 202; these dots are reflections of the features projected by the projector 116. This intensity of the reflections, and hence the change in surface, is monitored over time for the grid boxes in the ROI; in some embodiments, not all of the features in a grid box and/or not all of the grid boxes in the ROI are monitored. In some embodiments, each individual pixel can be considered an ROI or a subset of pixels can be identified and each subset considered an ROI.

In one embodiment, to determine a cardiac parameter of the patient based on the light intensity reflected, the first ROI 201, focused on bare skin, is monitored. Ballistic forces produced by the pumping action of the heart produce small body movements. As such, the skin surface undergoes minor translations and rotations relative to the camera, which in turn, induces changes in reflected light intensities due to the light dispersion characteristics of the skin. The ballistic induced movements also translate to materials covering the skin (e.g., clothing or a sheet) and can therefore be measured in a similar manner as the bare skin on the second ROI 202.

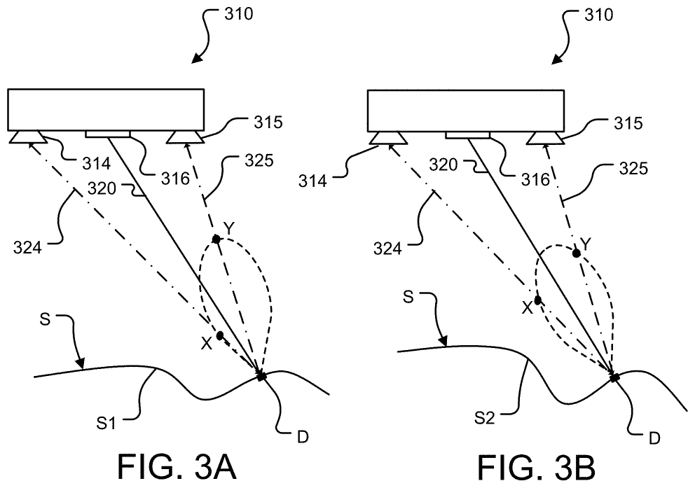

FIG. 3A and FIG. 3B illustrate the methodology for determining the light intensity of a projected and reflected IR feature and how that light intensity changes as the surface moves; this methodology generally applies to any projected and thus reflected feature, whether IR or another light source. The measurement of this reflected light intensity, or reflectance, is independent of any color change that may occur, thus distinguishing the methodology from PPG.

Both FIGS. 3A and 3B show a non-contact detector 310 having a first camera including an IR detection feature 314, a second IR camera including an IR detection feature 315, and an IR projector 316. A dot D is projected by the projector 316 onto a surface S, e.g., of a patient, via a beam 320. Light from the dot D is reflected by the surface S and is detected by the camera 314 as beam 324 and by the camera 315 as beam 325.

The light intensity returned to and observed by the cameras 314, 315 depends on the diffusion pattern caused by the surface S (e.g., the surface of a patient), the distance between the cameras 314, 315 and surface S, the surface gradient, and the orientation of the cameras 314, 315 relative to the surface S. In FIG. 3A, the surface S has a first profile S1 and in FIG. 3B, the surface S has a second profile S2 different than S1; as an example, the first profile S1 may be during the ventricular depolarization or atrial repolarization of a patient and the second profile S2 may be during ventricular repolarization (resting phase) of the patient. Because the surface profiles S1 and S2 differ, the deflection pattern from the dot D on each of the surfaces differs for the two figures.

The light intensity reflection off the dot D observed by the cameras 314, 315 changes because the surface profile S1 and S2 (specifically, the gradient) changes as well as the distance and orientation between the surface S and the cameras 314, 315. FIG. 3A shows the surface S having the surface profile S1 at time instant t=tₙ and FIG. 3B shows the surface S having the surface profile S2 at a later time, specifically t=tₙ₊₁, with S2 being slightly changed due to motion caused by movement of the skin. Consequently, the intensity of the dot D observed by the cameras 314, 315 will change due to the changes of the surface S. In FIG. 3A, a significantly greater intensity is measured by the camera 315 than the camera 314, seen by the x and y on the beams 324, 325, respectively. In FIG. 3B, y is less than y in FIG. 3A, whereas x in FIG. 3B is greater than x in FIG. 3A. The manner in how these intensities change depends on the diffusion pattern and its change over time. As seen in FIGS. 3A and 3B, the light intensities as measured by the cameras 314, 315 have changed between FIGS. 3A and 3B, and hence, the surface S has moved.

To produce a light intensity signal for the ROI (e.g., the ROI 201), in one embodiment, the light intensity of the reflected dots in each of the boxes in the grid pattern measured as described above is summed or averaged or weight averaged or any other method is used to combine the dots in each of the boxes. In some embodiments, less than all the reflected dots in the ROI 201 are monitored; for example, only a random sampling of the features is monitored, or for example, every third feature is monitored. In some embodiments, each feature reflection over time is monitored only for a predetermined duration, to determine which projected features provide an accurate or otherwise desired light intensity signal, and then those selected features are monitored to obtain the signal. In some embodiments, each pixel in the ROI 201 is monitored and the light intensity signal obtained.

It is noted that when two cameras 314, 315 are used, although both cameras will produce very similar results, they each have their own noise characteristics. The noise, which is added to the signal, is generally uncorrelated and the overall noise component is therefore reduced by combining the results of two cameras. Thus, each camera 314, 315 produces a signal pattern and the results may then be, for example, averaged. Note that other, more advanced, methods for combining/fusing the different signals may be used including Kalman and particle filtering. More than two cameras can also be used to even further reduce the noise in the signal.

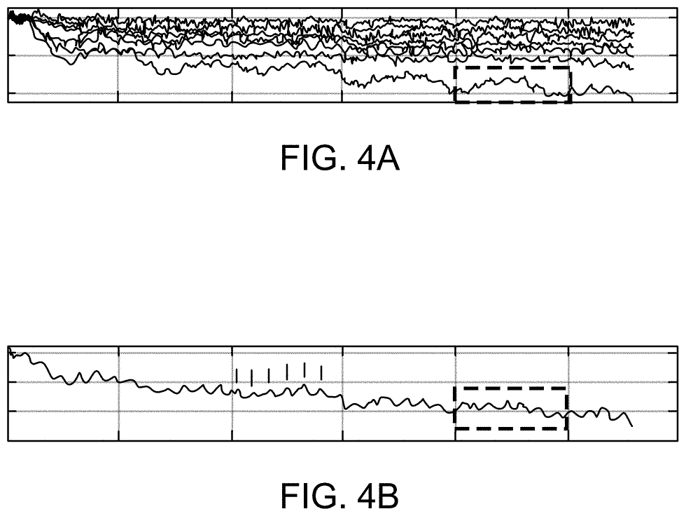

An example of multiple summed light intensity signals (individual dots in a grid being combined) for multiple grid boxes is shown in FIG. 4A; that is, individual light intensity signals within a grid were summed to provide the graph of FIG. 4A. Each line or signal represents the sum of individual light intensities in a grid box. The signals from the multiple grid boxes can be combined to produce a combined light intensity signal, an example of which is shown in FIG. 4B. The signals can be combined in any manner to produce the combined light intensity signal. These methods can include, but is not limited to, weighted average and/or Kalman and particle filtering.

In the graph of FIG. 4B, distinct peaks can be seen in the combined light intensity signal, some of which are called out by arrows. Each of these peaks represents a distinct pulse of blood through the grid box area, indicative of a heartbeat or pulse. By knowing the timing of the heartbeats, a heart rate can be derived.

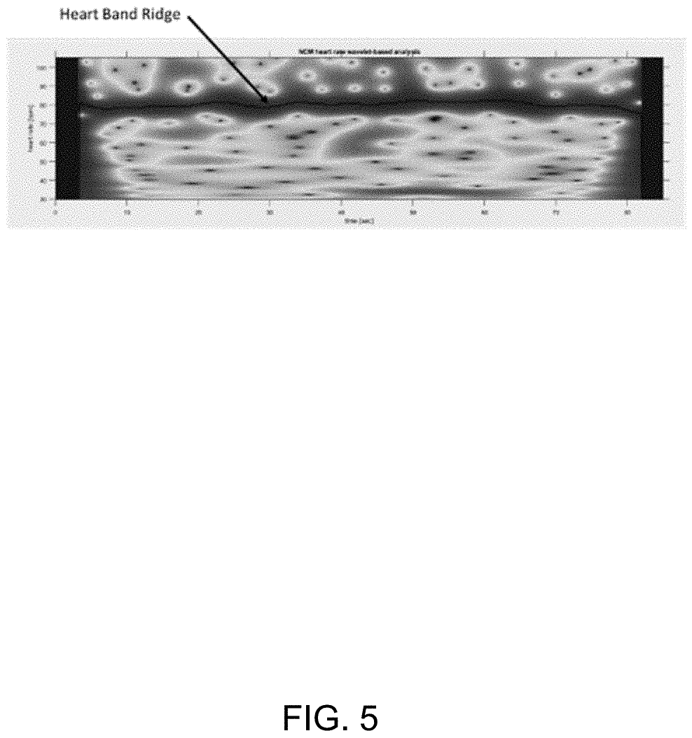

A heart rate can be derived from the signal of FIG. 4B using any of multiple methods. The most straightforward method is by counting the number of beats over a set period of time. Another method is by finding a distinct peak in a frequency spectrum within a region of an expected heart rate. Also, a time-frequency method can be used, such as a wavelet transform method, where the heart rate ridge is extracted from the heart band that runs across the transform plane to provide the heart rate over time (see, e.g., FIG. 5 where a heart band ridge is indicated). As another example, a weighted sum of the grid box signals (the signals shown in FIG. 4A) may be used to derive the heart rate, where the weight depends on, e.g., the quality of each signal, the intensity of each signal, the phase of each signal, etc. Another method to derive the heart rate is to find a heart rate for multiple signals (of FIG. 4A, by any manner) and combine those rates to determine the average heart rate; it is noted that distinct outliers can be excluded from this average.

For any of the above listed methods, high and/or low frequency components may be filtered from the signal before calculating the heart rate, as well as any other evident outliers. In some embodiments, the individual phases of any signal may be corrected or offset so that the signals are in phase before combining or averaging.

Across the whole ROI, some features (e.g., dots or each pixel) may produce “in phase” modulations and some may be “out of phase.” These may be combined separately to produce two signals. Light returning from each of these features may be combined to produce a single respiratory signal, for example by inverting or phase-shifting by 180 degrees so where necessary to produce all in phase and then combining to get a combined pattern signal.

Returning to FIG. 2, as described above, the ROI 201 on the patient's face, on bare skin, can be used to determine heart rate from the change in light intensity of the features projected in the ROI 201. A respiratory parameter, such as the respiratory rate, can also be derived using the same method as the heart rate. Either or both the ROI 201 and the ROI 202 can be used to determine respiratory information; as the ROI 202 is focused on the chest or torso of the patient, which typically moves in concert with the patient's respiration, the ROI 202 will be commonly used. However, depending on the orientation of the patient and their habits (e.g., breathing through their mouth), the face may also move with the patient's respiration; thus, ROI 201 could alternately be used.

From the graphs of FIGS. 4A and 4B, it is seen that a respiration signal is also present in the data. An example of a single respiratory modulation in the signal is highlighted within the dashed regions (e.g., rectangles) in FIG. 4A and FIG. 4B. The respiratory and heart rate signals can be separated or extracted through filtering, time-frequency methods, or any other method from the combined respiratory and heart signal. The new, separate, respiratory and heart rate signals can then be further analyzed to obtain the respiratory rate and heart rate respectively.

The light intensity reflection from the features in the selected grid boxes of the ROI can be used to determine a respiration parameter. Similar to the method described above for the cardiac parameter (e.g., heartbeat), the light intensity returned to and observed by the cameras 314, 315 depends on the diffusion pattern caused by the surface S (e.g., the surface of a patient), the distance between the cameras 314, 315 and surface S, the surface gradient, and the orientation of the cameras 314, 315 relative to the surface S. The movement of the surface can be indicative of respiration. As an example, the first profile S1 is during an exhale breath of a patient and the second profile S2 is during an inhale breath of the patient.

During breathing (respiration), as with detection of a heartbeat, the light intensity reflection off the dot D observed by the cameras 314, 315 changes because the surface profile S1 and S2 (specifically, the gradient) changes as well as the distance between the surface S and the cameras 314, 315. The intensity of the dot D observed by the cameras 314, 315 will change due to the changes of the surface S. From these intensity changes, the movement of the ROI (e.g., the patient's chest or torso or face) can be determined and applied to a respiration parameter.

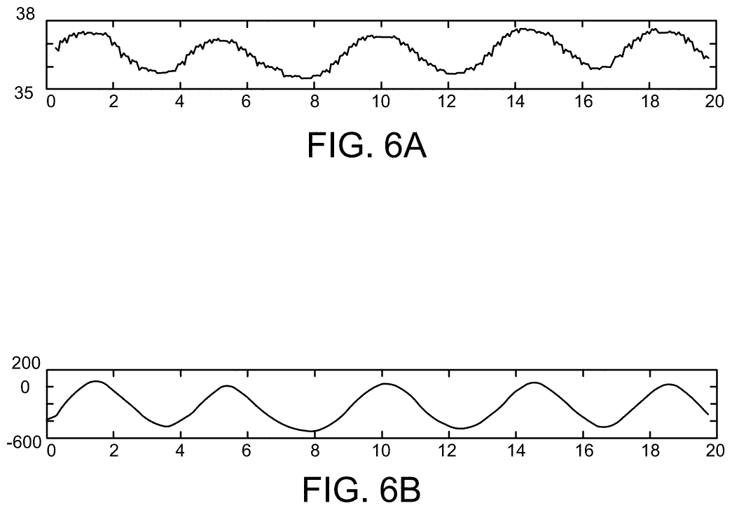

This change of intensity over time of each of the projected features can be used to produce a respiratory waveform plot. The waveform is formed by aggregating all of the intensity or pixel values, at an instant in time, over time, from across the ROI to generate a pattern signal, such as shown in FIG. 6A. In some embodiments, less than all the projected features in the ROI are monitored; for example, only a random sampling of the projected features is monitored, or for example, every third feature is monitored. In some embodiments, each feature reflection over time is monitored only for a predetermined duration, to determine which projected features provide an accurate or otherwise desired light intensity signal, and then those selected features are monitored to obtain the signal. In some embodiments, each pixel in the ROI is monitored and the light intensity signal obtained.

The methods of this disclosure additionally can utilize depth (distance) information between the camera(s) and the patient to determine a respiratory parameter such as respiratory rate. A depth image or depth map, which includes information about the distance from the camera to each point in the image, can be measured or otherwise captured by a depth sensing camera, such as a Kinect camera from Microsoft Corp. (Redmond, Wash.) or a RealSense™ D415, D435 or D455 camera from Intel Corp. (Santa Clara, Calif.) or other sensor devices based upon, for example, millimeter wave and acoustic principles to measure distance.

The depth image or map can be obtained by a stereo camera, a camera cluster, camera array, or a motion sensor focused on a ROI, such as a patient's chest. In some embodiments, the camera(s) are focused on visible or IR features in the ROI; these features may be the same as or different ones from those used for the light intensity. Each feature may be monitored, less than all the features in the ROI may be monitored or all the pixels in the ROI can be monitored.

When multiple depth images are taken over time in a video stream, the video information includes the movement of the points within the image, as they move toward and away from the camera over time.

Because the image or map includes depth data from the depth sensing camera, information on the spatial location of the patient (e.g., the patient's chest) in the ROI can be determined. This information can be contained, e.g., within a matrix. As the patient breathes, the patient's chest moves toward and away from the camera, changing the depth information associated with the images over time. As a result, the location information associated with the ROI changes over time. The position of individual points within the ROI (i.e., the change in distance from the monitoring system) may be integrated across the area of the ROI to provide a change in volume over time. For example, movement of a patient's chest toward a camera as the patient's chest expands forward represents inhalation. Similarly, movement backward, away from the camera, occurs when the patient's chest contracts with exhalation. This movement forward and backward can be tracked to determine a respiration rate. Additionally, the changes in the parameter can be monitored over time for anomalies, e.g., signals of sleep apnea or other respiratory patterns.

In some embodiments, the depth signal from the non-contact system may need to be calibrated, e.g., to provide an absolute measure of volume. For example, the volume signal obtained from integrating points in a ROI over time may accurately track a patient's tidal volume and may be adjusted by a calibration factor or factors. The calibration or correction factor could be a linear relationship such as a linear slope and intercept, a coefficient, or other relationships. As an example, the volume signal obtained from a video camera may under-estimate the total tidal volume of a patient, due to underestimating the volume of breath that expands a patient's chest backward, away from the camera, which is not measured by the depth cameras, or upward orthogonal to the line of sight of the camera. Thus, the non-contact volume signal may be adjusted by simply adding or applying a correction or calibration factor. This correction factor can be determined in a few different ways, including measuring the actual parameter to obtain a reference value to use as a baseline.

In some embodiments, demographic data about a patient may be used to calibrate the depth or volume signal. From a knowledge of the patient's demographic data, which may include height, weight, chest circumference, BMI, age, sex, etc., a mapping from the measured volume signal to an actual volume signal may be determined. For example, patients of smaller height and/or weight may have less of a weighting coefficient for adjusting measured volume for a given ROI box size than patients of greater height and/or weight. Different corrections or mappings may also be used for other factors, such as whether the patient is under bedding, type/style of clothing worn by a patient (e.g., t-shirt, sweatshirt, hospital gown, dress, v-neck shirt/dress, etc.), thickness/material of clothing/bedding, a posture of the patient, and/or an activity of the patient (e.g., eating, talking, sleeping, awake, moving, walking, running, etc.).

The respiratory modulations over time, extracted from the depth (distance) varying over time as measured by the depth camera(s) (shown in FIG. 6B), closely match the respiratory modulations obtained from the light intensity of the projected features (shown in FIG. 6A).

This method for producing a respiratory signal, i.e., from the depth data, is independent from the intensity of the light diffusion used to produce a signal representative of the respiratory parameter. This secondary pattern signal, from the depth, can be used to enhance or confirm the measurement of the respiratory parameters from the light intensity, and vice versa.

For example, the calculation of respiratory rate (determined from, e.g., a plot such as FIG. 6A created from the light intensity) can be combined with a similar plot of the respiratory rate obtained from the depth camera (e.g., FIG. 6B). This may be done, e.g., by computing respiratory rate from each signal and then averaging the two numbers, or, with a more advanced method, such as Kalman filtering.

It should be noted that the phase of the intensity pattern signal (FIG. 6A) may be 180 degrees out of phase with that of the depth signal (FIG. 6B). This would be due the direction of the movement of the surface (e.g., exhale versus inhale) and gradient of the surface as well as orientation of the camera(s) relative to the surface, all which play a role in modulating the reflected light.

Across the whole ROI, some features (e.g., dots or each pixel) may produce “in phase” modulations and some may be “out of phase.” These may be combined separately to produce two signals. Light returning from each of these features may be combined to produce a single respiratory signal, for example by inverting or phase-shifting by 180 degrees so where necessary to produce all in phase and then combining to get a combined pattern signal.

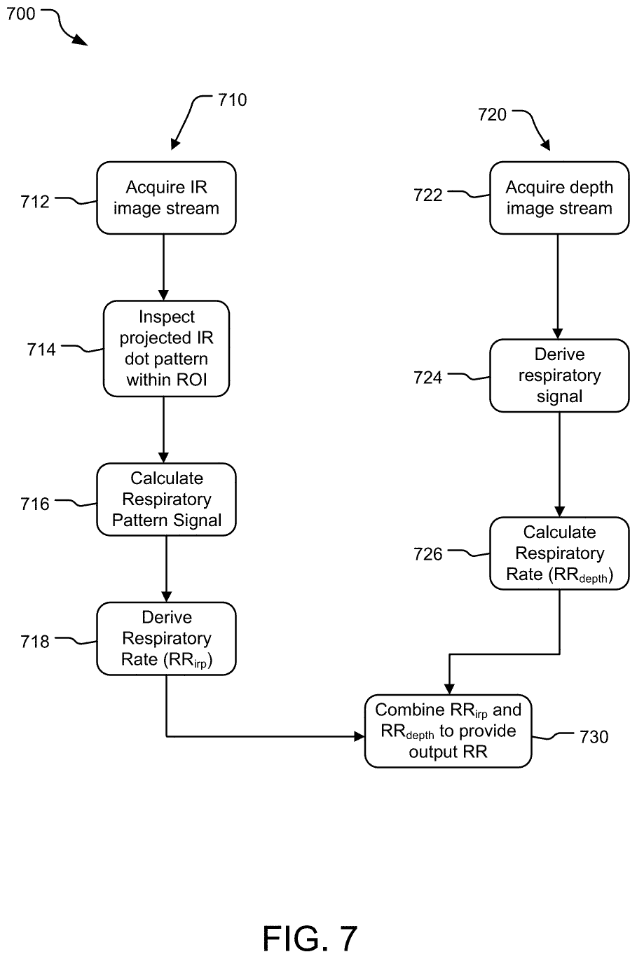

FIG. 7 shows a method 700 for combining the data from the depth measurements with the data from the light intensity measurements to provide a combined respiratory parameter. In FIG. 7, the method 700 is particularly directed to respiratory rate, whereas in other embodiments a similar method is used to provide a different respiratory parameter.

The method 700 includes a first branch 710 that derives a respiratory parameter (specifically for this example, the respiratory rate (RRᵢᵣₚ)) from the light intensity measurements and a second branch 720 that calculates the respiratory parameter (specifically for this example, the respiratory rate (RR_{depth})) from the depth measurements. The method 700 combines the derived respiratory rate (RRᵢᵣₚ) from the first branch 710 with the calculated respiratory rate (RR_{depth}) from the second branch 720.

For the respiratory rate (RRᵢᵣₚ) derived from the light intensity measurements, the method 700 includes a step 712 where the IR images are acquired of the surface being monitored. The features within the desired ROI are inspected in step 714 for their light intensity and change in light intensity over time. From the intensity information obtained in step 714, a respiratory pattern signal is calculated in step 716. From this patterned signal, the respiratory rate (RRᵢᵣₚ) is derived.

For the respiratory rate (RR_{depth}) derived from the depth measurements, the method 700 includes step 722 where the depth image stream of the surface is acquired from a depth camera. A respiratory signal (e.g., volume) is derived from the depth stream in step 724, from which a respiratory rate (RR_{depth}) is calculated in step 726.

In step 730, the derived respiratory rate (RRᵢᵣₚ) from step 718 is combined with the respiratory rate (RR_{depth}) calculated in step 726. The two rates may be averaged (e.g., simple average or mean, median, etc.), added, or combined in any other manner. Either individual patterns or the combined pattern can be inspected for anomalies, e.g., signals of sleep apnea or other respiratory patterns.

Other methods for combining the calculation of respiratory rate (determined from light intensity, e.g., a plot such as FIG. 6A) with a similar plot of the respiratory rate obtained from the depth camera (e.g., a plot such as FIG. 6B) can be used.

Any or all of the parameters, i.e., the cardiac information determined by light intensity, the respiratory information determined by light intensity, and the respiratory information determined by depth information, may be measured and/or calculated simultaneously or sequentially.

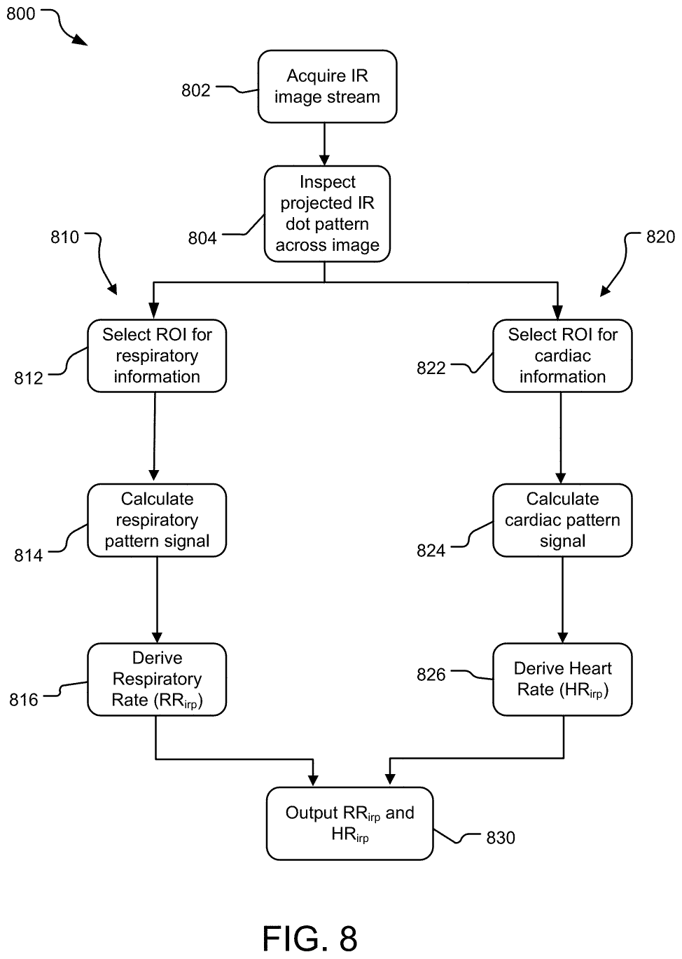

FIG. 8 shows a flow chart of a method 800 for simultaneously determining both a respiratory rate and a heart rate (e.g., by the computing device 120) simultaneously with the data being received from the detector system 110. This method 800 is particularly directed to utilizing projected IR features and the reflected light intensity therefrom, although a same method can be used for other sources of projected features.

In a first step 802, the IR images are acquired of the surface being monitored. The features (e.g., dots) within the desired ROI are inspected in step 804 for their light intensity and change in light intensity over time.

For the respiratory parameter, the method 800 includes a first branch 810 that derives a respiratory parameter (specifically for this example, the respiratory rate (RRᵢᵣₚ)) from the light intensity measurements and a second branch 820 that derives a cardiac parameter (specifically for this example, the heart rate (HRᵢᵣₚ)) from the light intensity measurements. The method 800 combines the derived respiratory rate (RRᵢᵣₚ) from the first branch 810 with the derived heart rate (HRᵢᵣₚ) from the second branch 820 for a simultaneous output.

Specifically, in branch 810, an appropriate ROI for respiratory information is selected in step 812; an appropriate ROI may be, e.g., the torso, chest, or mouth/nose region of the patient. The light intensity reflecting in the ROI is monitored and the respiratory pattern signal is calculated therefrom in step 814. From the pattern, the respiratory rate (RRᵢᵣₚ) is derived in step 816.

Simultaneously in branch 820, an appropriate ROI for cardiac information is selected in step 822; an appropriate ROI may be, e.g., the forehead or cheeks region of the patient. The light intensity reflecting in the ROI is monitored and a cardiac pattern signal is calculated therefrom in step 824. From the pattern, the heart rate (HRᵢᵣₚ) is derived in step 826.

The two rates, the respiratory rate (RRᵢᵣₚ) and the heart rate (HRᵢᵣₚ), are each output in step 830.

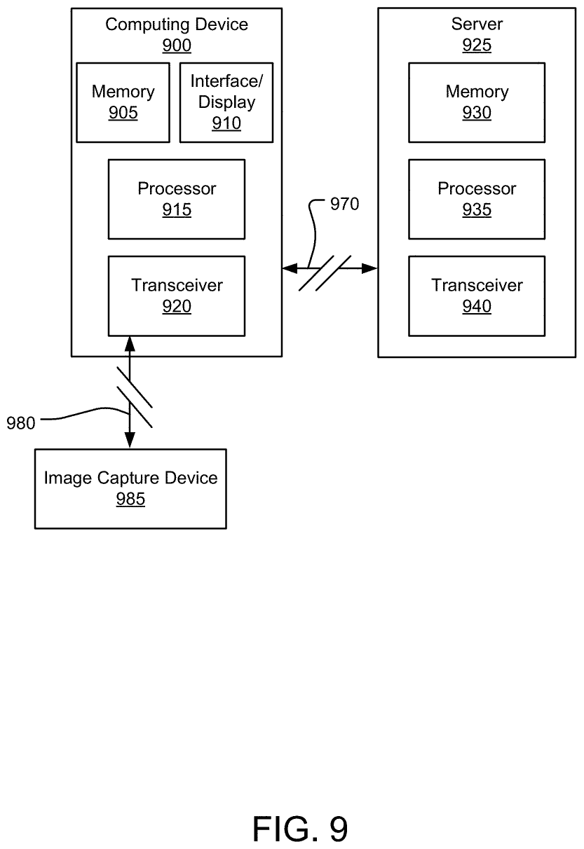

FIG. 9 is a block diagram illustrating a system including a computing device 900, a server 925, and an image capture device 985 (e.g., the cameras 114, 115 or the cameras 314, 315). In various embodiments, fewer, additional and/or different components may be used in the system.

The computing device 900 includes a processor 915 that is coupled to a memory 905. The processor 915 can store and recall data and applications in the memory 905, including applications that process information and send commands/signals according to any of the methods disclosed herein. The processor 915 may also display objects, applications, data, etc. on an interface/display 910. The processor 915 may also or alternately receive inputs through the interface/display 910. The processor 915 is also coupled to a transceiver 920. With this configuration, the processor 915, and subsequently the computing device 900, can communicate with other devices, such as the server 925 through a connection 970 and the image capture device 985 through a connection 980. For example, the computing device 900 may send to the server 925 information determined about a patient from images captured by the image capture device 985, such as depth information of a patient in an image.

The server 925 also includes a processor 935 that is coupled to a memory 930 and to a transceiver 940. The processor 935 can store and recall data and applications in the memory 930. With this configuration, the processor 935, and subsequently the server 925, can communicate with other devices, such as the computing device 900 through the connection 970.

The computing device 900 may be, e.g., the computing device 120 of FIG. 1. Accordingly, the computing device 900 may be located remotely from the image capture device 985, or it may be local and close to the image capture device 985 (e.g., in the same room). The processor 915 of the computing device 900 may perform any or all of the various steps disclosed herein. In other embodiments, the steps may be performed on a processor 935 of the server 925. In some embodiments, the various steps and methods disclosed herein may be performed by both of the processors 915 and 935. In some embodiments, certain steps may be performed by the processor 915 while others are performed by the processor 935. In some embodiments, information determined by the processor 915 may be sent to the server 925 for storage and/or further processing.

The devices shown in the illustrative embodiment may be utilized in various ways. For example, either or both of the connections 970, 980 may be varied. For example, either or both the connections 970, 980 may be a hard-wired connection. A hard-wired connection may involve connecting the devices through a USB (universal serial bus) port, serial port, parallel port, or other type of wired connection to facilitate the transfer of data and information between a processor of a device and a second processor of a second device. In another example, one or both of the connections 970, 980 may be a dock where one device may plug into another device. As another example, one or both of the connections 970, 980 may be a wireless connection. These connections may be any sort of wireless connection, including, but not limited to, Bluetooth connectivity, Wi-Fi connectivity, infrared, visible light, radio frequency (RF) signals, or other wireless protocols/methods. For example, other possible modes of wireless communication may include near-field communications, such as passive radio-frequency identification (RFID) and active RFID technologies. RFID and similar near-field communications may allow the various devices to communicate in short range when they are placed proximate to one another. In yet another example, the various devices may connect through an internet (or other network) connection. That is, one or both of the connections 970, 980 may represent several different computing devices and network components that allow the various devices to communicate through the internet, either through a hard-wired or wireless connection. One or both of the connections 970, 980 may also be a combination of several modes of connection.

The configuration of the devices in FIG. 9 is merely one physical system on which the disclosed embodiments may be executed. Other configurations of the devices shown may exist to practice the disclosed embodiments. Further, configurations of additional or fewer devices than the ones shown in FIG. 9 may exist to practice the disclosed embodiments. Additionally, the devices shown in FIG. 9 may be combined to allow for fewer devices than shown or separated such that more than the three devices exist in a system. It will be appreciated that many various combinations of computing devices may execute the methods and systems disclosed herein. Examples of such computing devices may include other types of medical devices and sensors, infrared cameras/detectors, night vision cameras/detectors, other types of cameras, radio frequency transmitters/receivers, smart phones, personal computers, servers, laptop computers, tablets, RFID enabled devices, or any combinations of such devices.

Thus, described herein are methods and systems for non-contact monitoring of a patient to determine cardiac parameters and respiratory parameters. The methods and systems of this disclosure utilize light intensity of reflected features (e.g., IR, UV, RBG, or other light) to determine cardiac parameter such as heart rate or pulse and also a respiratory parameter such as respiratory rate. The methods and systems of this disclosure additionally can utilize depth (distance) information between the camera(s) and the patient to determine a respiratory parameter such as respiratory rate.

The above specification and examples provide a complete description of the structure and use of exemplary implementations of the invention. The above description provides specific implementations and embodiments. It is to be understood that other implementations and embodiments are contemplated and may be made without departing from the scope or spirit of the present disclosure. The above detailed description, therefore, is not to be taken in a limiting sense. For example, elements or features of one example, embodiment or implementation may be applied to any other example, embodiment or implementation described herein to the extent such contents do not conflict. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about,” whether or not the term “about” is immediately present. Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

1. A method of monitoring a patient by a non-contact patient monitoring system in a region of interest (ROI), over time, the method comprising:
determining a cardiac parameter of the patient using reflected light intensity information by:
projecting a light feature onto a surface of the patient in the ROI;
measuring a first reflected light intensity from the light feature at a first time;
measuring a second reflected light intensity from the light feature at a second time subsequent to the first time;
comparing the first reflected light intensity and the second reflected light intensity to determine a change in profile of the surface over time; and
correlating the change to the cardiac parameter.

2. The method of claim 1, wherein correlating the change to the cardiac parameter comprises:
obtaining a pattern in the change in profile of the surface over time; and
correlating the pattern to the cardiac parameter.

3. The method of claim 1, wherein the cardiac parameter is heart rate.

4. The method of claim 1, wherein projecting the light feature comprises:
projecting an IR light feature.

5. The method of claim 1, wherein projecting the light feature comprises:
projecting the light feature on a forehead of the patient.

6. The method of claim 1, wherein projecting the light feature onto the surface of the patient in the ROI comprises:
projecting a plurality of light features onto the surface of the patient in the ROI.

7. The method of claim 1, wherein determining the cardiac parameter using reflected light intensity information comprises:
measuring the first reflected light intensity and the second reflected light intensity from the light feature in stereo with a first camera and a second camera.

8. The method of claim 7, wherein measuring with the first camera and the second camera comprises:
comparing the first reflected light intensity measured by the first camera and the second camera to the second reflected light intensity measured by the first camera and the second camera.

9. The method of claim 1 further comprising:
determining a respiratory parameter of the patient using a second reflected light intensity information in a second ROI, over time, from the patient, determined by:
projecting a second light feature onto the surface of the patient in the second ROI over time;
measuring a third reflected light intensity from the second light feature at a third time;
measuring a fourth reflected light intensity from the second light feature at a fourth time subsequent to the third time;
comparing the third reflected light intensity and the fourth reflected light intensity to determine a second change in profile of the surface over time; and
correlating the second change to the respiratory parameter.

10. The method of claim 9, wherein correlating the second change to the respiratory parameter comprises:
obtaining a second pattern in the second change in profile of the surface over time; and
correlating the second pattern to the respiratory parameter.

11. The method of claim 9, wherein the second ROI is different from the ROI.

12. The method of claim 9, wherein the second ROI is the same as the ROI.

13. The method of claim 9, wherein the third time is the same as the first time and the fourth time is the same as the second time.

14. The method of claim 9, wherein the third time is different from the first time and the fourth time is different from the second time.

15. The method of claim 9 further comprising:
determining a respiratory parameter of the patient using depth information.

16. A method of monitoring a patient by a non-contact patient monitoring system in a region of interest (ROI), over time, the method comprising:
determining a cardiac parameter of the patient using light reflectance information by:
measuring a first light reflectance from a projected IR light feature at a first time in the ROI, the projected IR light feature having been projected onto a surface of the patient;
measuring a second light reflectance from the projected IR light feature at a second time in the ROI, wherein the second time is subsequent to the first time;
comparing the first light reflectance and the second light reflectance to determine a change in the surface over time; and
correlating the change to the cardiac parameter of the patient.

17. The method of claim 16 further comprising:
obtaining a pattern in the change in the surface over time; and
correlating the pattern to the cardiac parameter.

18. The method of claim 16, wherein measuring the first light reflectance and measuring the second light reflectance comprises:
measuring the first light reflectance and the second light reflectance from the projected IR light feature in stereo with a first camera and a second camera.

19. The method of claim 16 further comprising:
determining a respiratory parameter of the patient using light reflectance information by:
measuring a third light reflectance from the light feature in the ROI at a third time;
measuring a fourth light reflectance from the light feature in the ROIL at a fourth time subsequent to the third time;
comparing the third light reflectance and the fourth light reflectance to determine a second change in profile of the surface over time; and
correlating the change to the respiratory parameter.

20. The method of claim 19 further comprising:
determining the respiratory parameter of the patient using depth information; and
comparing the respiratory parameter determined using depth information to the respiratory parameter determined using light reflectance.
